(19) United States
(12) Patent Application Publication
(10) Pub. No.: US 2016/0074449 A1
(43) Pub. Date: Mar. 17, 2016

(54) Food Composition for Increased Breast Milk Production

(71) Applicant: Tanyika Miller, Zephyrhills, FL (US)

(72) Inventor: Tanyika Miller, Zephyrhills, FL (US)

(21) Appl. No.: 14/857,358

(22) Filed: Sep. 17, 2015

Related U.S. Application Data
(60) Provisional application No. 62/051,455, filed on Sep. 17, 2014.

(51) Int. Cl. A61K 36/064; A61K 36/889; A21D 8/04; A61K 9/00; A21D 13/00; A61K 36/55; A61K 47/46

(52) U.S. Cl. CPC A61K 36/064; A61K 36/55; A61K 36/889; A61K 47/46; A61K 9/0056; A21D 13/0067; A21D 8/047

(57) ABSTRACT

A baked good that promotes the production of breast milk in a nursing mother. The present food composition is intended to increase the production of milk while being presented in a baked form. It is prepared with brewer's yeast, vanilla extract, and water. The composition is further intended to be prepared by means of baking the ingredients in an oven, so that the final food composition is in a solid form. There are several embodiments of the present invention, including in a cookie, muffin, and brownie form.

### CROSS REFERENCE TO RELATED APPLICATIONS

This application claims the benefit of U.S. Provisional Application No. 62/051,455 filed on Sep. 17, 2014. The above identified patent application is herein incorporated by reference in its entirety to provide continuity of disclosure.

### FIELD OF THE INVENTION

The present invention relates to a composition of food. More specifically, the present invention relates to a composition of food that is formulated to stimulate production of breast milk in a nursing mother.

### BACKGROUND OF THE INVENTION

Nursing an infant with mother's breast milk is a natural, nutritious, and convenient method of feeding a baby. Because of the many advantages breast milk offers over baby formula, many mothers prefer to give their newborn breast milk, either through nursing or through expressed breast milk via pumping, rather than provide them with bottles of formula. However, some women may be unable to produce a sufficient amount of breast milk, either in the initial stages of breastfeeding or at later stages of child development.

While some food products are known to increase breast milk production, many products do not do so efficiently. Furthermore, many products that do offer increased milk production do not having a pleasing taste. Composition in the prior art specifically related to increasing lactation production generally relate to additives to be combined with other foods, or to compositions in a liquid form comprising various ingredients, such as vitamins and minerals. These compositions, however, do not provide a composition for producing baked goods containing a combination of brewer's yeast, vanilla extract, and water.

### SUMMARY OF THE INVENTION

In view of the foregoing disadvantages inherent in the known types of food compositions made to promote breast milk production now present in the prior art, the present invention provides a food composition wherein the same can be formulated to be prepared in the form of a pleasantly tasting baked good. In particular, brewer's yeast is used to promote the increase in breast milk production. However, the yeast also introduces a bitter taste into the mixture. To counter this undesirable side effect, vanilla extract is added to mask the bitterness and give the product an appealing taste. Various other ingredients are added in different embodiments of the invention to improve the taste, including coconut oil, canola oil and flax seed.

### DETAILED DESCRIPTION OF THE INVENTION

The present invention is a baked good that encourages increased milk production. The active ingredients include brewer's yeast, a fungus which is rich in vitamins and the element chromium. While the brewer's yeast is effective in increasing lactation production, it also introduces a bitter taste into a food mixture when added. In order to neutralize this bitter taste, pleasant tasting ingredient may be added, so long as they do not inhibit the positive effects of the brewer's yeast. One embodiment of the present invention is presented in the form of a baked sweet good, such as a cookie, comprising active ingredients of 1 part brewer's yeast, 1 part flax seed, 2 parts coconut oil, 1 part vanilla extract, and 4 part water, which is combined with flour, sugar, oats, vegetable oil and a leavening agent such as baking soda. The mixture is subsequently cooled inside a refrigerator until it is set and then baked in an oven for approximately 8-14 minutes at 325-375 degrees Fahrenheit.

Another embodiment of the current invention is a baked sweet good, such as a muffin, comprising active ingredients of 1 part brewer's yeast, 1 part vanilla extract, and 4 parts water, which is combined with flour, sugar, dextrose, and vegetable oil. The mixture is subsequently baked in an oven for approximately 11 minutes at 325 to 375 degrees Fahrenheit.

Yet another embodiment of the current invention is a baked sweet good, such as a brownie, comprising active ingredients of 3 part brewer's yeast, 3 part vanilla extract, 16 parts canola oil, 12 parts water, and 2 eggs, which is combined with flour, sugar, cocoa, corn syrup, vegetable oil and corn starch. The mixture is subsequently baked in an oven for approximately 38 minutes at 325 to 375 degrees Fahrenheit.

It is therefore submitted that the instant invention has been shown and described in what is considered to be the most practical and preferred embodiments. It is recognized, however, that departures may be made within the scope of the invention and that obvious modifications will occur to a person skilled in the art.

Therefore, the foregoing is considered as illustrative only of the principles of the invention. Further, since numerous modifications and changes will readily occur to those skilled in the art, it is not desired to limit the invention to the exact composition described, and accordingly, all suitable modifications and equivalents may be resorted to, falling within the scope of the invention.

I claim:

1. A baked good, comprising:
1 part brewer's yeast;
1 part flax seed;
2 parts coconut oil;
1 part vanilla extract;
4 parts water.

2. A method of preparing the baked good claimed of claim 1, wherein the ingredients are combined, placed in an area to cool and set, and subsequently baked at about 325-375 degrees Fahrenheit for about 8-14 minutes.

3. A baked good, comprising:
1 part brewer's yeast;
1 part vanilla extract;
4 parts water.

4. A method of preparing the baked good claimed of claim 3, wherein the ingredients are combined and subsequently baked at about 400-450 degrees Fahrenheit for about 8-14 minutes.

5. A baked good, comprising:
3 part brewer's yeast;
3 part vanilla extract;
16 parts canola oil;
12 parts water;
2 eggs.

6. A method of preparing the baked good claimed of claim 5, wherein the ingredients are combined and subsequently baked at about 300-350 degrees Fahrenheit for about 33-43 minutes.
